(19) United States
(12) Patent Application Publication
(10) Pub. No.: US 2024/0277616 A1
(43) Pub. Date: Aug. 22, 2024

(54) Lipid Nanoparticle-Mediated mRNA Delivery to the Pancreas

(71) Applicant: Carnegie Mellon University, Pittsburgh, PA (US)

(72) Inventors: Kathryn Ann Whitehead, Pittsburgh, PA (US); Jilian Melamed, Pittsburgh, PA (US)

(21) Appl. No.: 18/289,661

(22) PCT Filed: May 6, 2022
(86) PCT No.: PCT/US22/28131

§ 371 Date: Nov. 6, 2023

Related U.S. Application Data
(60) Provisional application No. 63/185,535, filed on May 7, 2021.

(51) Int. Cl. A61K 9/127; A61K 9/51; A61K 31/711

(52) U.S. Cl. CPC A61K 9/1272; A61K 9/5123; A61K 31/711

(57) ABSTRACT

Lipid-containing particles and formulations containing the lipid-containing particles are provided. Methods of delivery of therapeutic agents to the pancreas using the lipid-containing particles are provided.

### CROSS REFERENCE TO RELATED APPLICATION

This Application claims the benefit of U.S. Provisional Patent Application No. 63/185,535 filed May 7, 2021, the disclosure of which is incorporated herein by reference in its entirety.

### STATEMENT REGARDING FEDERAL FUNDING

This invention was made with United States government support under Grant Nos. EB029345 and HD098860, awarded by the National Institutes of Health, and Grant No. D16AP00143, awarded by the Defense Advanced Research Projects Agency. The U.S. government has certain rights in the invention.

### REFERENCE TO A SEQUENCE LISTING SUBMITTED VIA EFS-WEB

The Sequence Listing associated with this application is filed in electronic format via EFS-Web and is hereby incorporated by reference into the specification in its entirety. The name of the text file containing the Sequence Listing is 2202416_ST25.txt. The size of the text file is 24,662 bytes, and the text filed was created on May 3, 2022.

Lipid nanoparticles are efficient carriers of cargo, such as a nucleic acid cargo, for gene delivery, mRNA delivery, antisense, RNA interference, among other uses. Lipid nanoparticles typically comprise helper lipids, cholesterol, ionizable lipids (e.g., lipidoids), lipid-polymer conjugates, and nucleic acid cargo. Lipid nanoparticles are typically administered in an intravenous, intramuscular, or subcutaneous injection. Exemplary LNP compositions and/or compositions, e.g., lipidoids, useful in producing LNPs are described in U.S. Pat. Nos. 10,844,028, 10,189,802, 9,872,911, 9,556,110, 9,439,968, 9,227,917, 8,969,353, and 8,450,298, as well as in U.S. Patent Application Publication Nos. 2017/0204075, 2019/0177289, 2017/0152213, 2016/0114042, 2015/0203439, 2014/0322309, 2014/0161830, 2011/0293703, and 2010/0331234, each of which incorporated herein by reference for its technical disclosure relating to compounds and compositions useful in delivery of nucleic acid cargoes, and to the extent it is consistent with the present disclosure. Additional examples of lipid nanoparticles are described in U.S. Pat. Nos. 9,404,127, 9,364,435, and 8,058,069, each of which incorporated herein by reference for its technical disclosure relating to compounds and compositions useful in delivery of nucleic acid cargoes, and to the extent it is consistent with the present disclosure (see, e.g., Sabnis S, et al., “A Novel Amino Lipid Series for mRNA Delivery: Improved Endosomal Escape and Sustained Pharmacology and Safety in Non-human Primates”, Mol Ther. 2018, 26(6): 1509-1519 and Yonezawa S, et al., “Recent advances in siRNA delivery mediated by lipid-based nanoparticles”, Adv Drug Deliv Rev. 2020; 154-155:64-78). Examples of lipid nanoparticles, lipidoids, and methods of making lipid nanoparticles and lipidoids are described in Whitehead K A, et al., “Degradable lipid nanoparticles with predictable in vivo siRNA delivery activity”, Nat Commun. 2014, 5:4277.

Despite some degree of success in parenteral delivery of nucleic acids via LNPs, there are significant obstacles to facile local delivery of nucleic acids, specifically to the pancreas.

### SUMMARY

Herein a method to mRNA delivery to the pancreas using lipid nanoparticles is described. In this method, lipid nanoparticles containing five components: a cationic helper lipid, cholesterol or a derivative thereof, a PEG-based compound, such as a PEG-containing polymer or PEGylated fatty acid-containing compound, an ionizable lipidoid, and mRNA were formulated. Delivering these particles to mice via intraperitoneal injection induces mRNA translation and, thus, protein expression specifically in the pancreas. Expression persists for at least 48 hours. This method also enables the simultaneous delivery of at least three mRNAs to the pancreas from a single lipid nanoparticle formulation, enabling delivery and relative dosing of multiple therapeutic polypeptides to a single cell.

A method of delivering a therapeutic agent to a pancreas of a patient is provided. The method comprises administering to a patient a composition comprising a lipid-containing particle, such as a lipid nanoparticle, comprising a therapeutic agent, the lipid-containing particle comprising:
a cationic helper lipid;
cholesterol or a derivative thereof;
a PEG-based compound, such as a PEG-containing polymer or a PEGylated fatty acid-containing compound; and
an ionizable lipidoid, e.g., that forms a cation at an acidic pH.

Non-limiting aspects or embodiments of the present invention will now be described in the following numbered clauses:
Clause 1: A method of delivering a therapeutic agent to a pancreas of a patient, comprising administering to a patient a composition comprising a lipid-containing particle, such as a lipid nanoparticle, comprising a therapeutic agent, the lipid-containing particle comprising: a cationic helper lipid; cholesterol or a derivative thereof; a PEG-based compound, such as a PEG-containing polymer or a PEGylated fatty acid-containing compound; and an ionizable lipidoid, e.g., that forms a cation at an acidic pH.
Clause 2: The method of clause 1, wherein the lipid particle comprises: from 10 to 50 mole percent (mol %) of the cationic helper lipid; from 10 to 46.5 mol % of the cholesterol or a derivative thereof; from 1.25 to 2.5 mol % of the PEG-based compound; and from 20 to 45 mol % of the ionizable lipidoid.
Clause 3: The method of clause 2, wherein the lipid particle comprises from 30 to 50 mol % of the cationic helper lipid.
Clause 4: The method of any of clauses 1 to 3, wherein the cationic helper lipid is one or more of 1,2-di-O-octadecenyl-3-trimethylammonium propane (DOTMA); a glycero-ethylphosphocholine (EPC) lipid, such as 12:0-sn-glycero-3-ethylphosphocholine (12:0 EPC), 14:0 EPC, 16:0 EPC, 18:0 EPC, 18:1 EPC, 16:0-18:1 EPC, and 14:1-EPC; dimethyldioctadecylammonium (DDAB); a trimethylammonium-propane (TAP) lipid, such as 14:0 TAP, 18:0 TAP, or 18:1 TAP (DOTAP); 3β-[N-(N′,N′-dimethylaminoethane)-carbamoyl]cholesterol hydrochloride (DC-chol); N⁴-cholesteryl-spermine (GL67); 1,2-dioleyloxy-3-dimethylaminopropane (DODMA); 1,2-dioleoyl-3-dimethylammonium-propane (DODAP); and N1-[2-((1S)-1-[(3-aminopropyl)amino]-4-[di(3-amino-propyl)amino] butylcarboxamido) ethyl]-3,4-di[oleyloxy]-benzamide (MVL5).
Clause 5: The method of clause 4, wherein the cationic helper lipid is DOTAP, EPC, or DDAB.
Clause 6: The method of any one of clauses 1 to 5, wherein the PEG-based compound is a PEGylated C₁₀-C₁₆ fatty acid-containing compound.
Clause 7: The method of clause 6, wherein the PEGylated fatty-containing compound is C₁₄-PEG₂₀₀₀ PE.
Clause 8: The method of any one of clauses 1 to 7, comprising cholesterol.
Clause 9: The method of any one of clauses 1 to 8, wherein the ionizable lipidoid is 306Oᵢ₁₀, 200Oᵢ₁₀, or 514O_{6,10}.
Clause 10: The method of any one of clauses 1 to 8, wherein the ionizable lipidoid is 306Oᵢ₁₀.
Clause 11: The method of any one of clauses 1 to 10, wherein the therapeutic agent is anionic or polyanionic.
Clause 12: The method of clause 11, wherein the therapeutic agent is a nucleic acid.
Clause 13: The method of clause 12, wherein the nucleic acid is an RNA.
Clause 14: The method of clause 13, wherein the RNA is an RNA reagent chosen from an RNAi reagent, a dsRNA, an siRNA, an shRNA, a miRNA, an antisense RNA, a guide RNA (gRNA), a long non-coding RNAs (IncRNA), a base editing gRNA (beRNA), a prime editing gRNA (pegRNA), or a transfer RNA (tRNA).
Clause 15: The method of clause 13, wherein the RNA is an mRNA.
Clause 16: The method of any one of clauses 1 to 15, wherein the lipid-containing particle comprises: DOTAP as the cationic helper lipid; cholesterol; C₁₄-PEG₂₀₀₀ PE as the PEGylated fatty acid-containing compound; and 306Oᵢ₁₀ as the ionizable lipidoid.
Clause 17: The method of any one of clauses 1 to 16, wherein the lipid-containing particle comprises the ionizable lipidoid, DOTAP, cholesterol, and C₁₄-PEG₂₀₀₀ PE in an ionizable lipidoid: DOTAP:cholesterol: C₁₄-PEG₂₀₀₀ PE molar ratio of less than or equal to 20 to less than or equal to 55: less than or equal to 10 to less than or equal to 60: less than or equal to 10 to less than or equal to 50: less than or equal to 1 to less than or equal to 2.5.
Clause 18: The method of clause 17, wherein the lipid-containing particle comprises the ionizable lipidoid, DOTAP, cholesterol, and C₁₄-PEG₂₀₀₀ PE in an ionizable lipidoid:DOTAP:cholesterol:C₁₄-PEG₂₀₀₀ PE molar ratio of approximately 35:40:22.5:2.5.
Clause 19: The method of any one of clauses 1 to 18, wherein the lipid-containing particle is delivered to the patient parenterally.
Clause 20: The method of clause 19, wherein the lipid-containing particle is delivered to the patient intravenously, intraperitoneally, intramuscularly, subcutaneously, or intradermally.
Clause 21: The method of any one of clauses 1 to 18, wherein the lipid-containing particle is delivered to the patient orally.
Clause 22: The method of any one of clauses 1 to 15, wherein the lipid-containing particle comprises: EPC as the cationic helper lipid; cholesterol; C₁₄-PEG₂₀₀₀ PE as the PEGylated fatty acid-containing compound; and 306Oᵢ₁₀ as the ionizable lipidoid.
Clause 23: The method of clause 22, wherein the EPC is 18:1 EPC.
Clause 24: The method of any one of clauses 1 to 15, wherein the lipid-containing particle comprises: DDAB as the cationic helper lipid; cholesterol; C₁₄-PEG₂₀₀₀ PE as the PEGylated fatty acid-containing compound; and 306Oᵢ₁₀ as the ionizable lipidoid.

### BRIEF DESCRIPTION OF THE DRAWINGS

FIGS. 1A and 1B are reaction schemes. FIG. 1A provides a general reaction scheme between an amine, e.g., the numbered compounds shown in FIGS. 2A-2C, and an acrylate tail having a carbon chain (Cₓ), e.g., the compounds designated O_, as shown in FIGS. 3A-3C to form a lipidoid, which is referenced by the reacted amine and the reacted acrylate tail (###O_), e.g., 306O₁₀, as shown in FIG. 1B. The compounds may be prepared by the addition of a primary or secondary amine to an acrylate via a Michael addition reaction.

FIGS. 2A-2C provide exemplary amines for use in preparing lipidoids as described herein. Each amine includes a reference number for use in the lipidoid designation.

FIGS. 3A-3C provide exemplary acrylates for use in preparing lipidoids as described herein.

FIGS. 4A-4C include bar graphs depicting the total luminescence and pie charts depicting the expression distribution in the lungs, liver, spleen, and pancreas after intraperitoneal (IP) and intravenous (IV) delivery of lipid nanoparticles. The lipid nanoparticles were prepared using 306Oᵢ₁₀ (FIG. 4A), 200Oᵢ₁₀ (FIG. 4B), and 514O_{6,10} (FIG. 4C) as the ionizable lipidoids.

FIGS. 5A-5F are bar graphs that depict total luminescence in the liver, lungs, spleen, and pancreas (FIGS. 5A, 5C, and 5E) and resulting signal in the pancreas (FIGS. 5B, 5D, and 5F) for lipid nanoparticles delivered intraperitoneally. The lipid nanoparticles were prepared using 306Oᵢ₁₀ (FIGS. 5A and 5B), 200Oᵢ₁₀ (FIGS. 5C and 5D), and 514O_{6,10} (FIGS. 5E and 5F) lipidoids with DOPE, PS, or DOTAP as the cationic helper lipid.

FIGS. 6A and 6B are graphs showing the persistence of luciferase mRNA expression, as measured by luminescence, in the pancreas, spleen, liver, and lungs for mice treated as described in Example 3 (FIG. 6A), and AUC (area under the curve) analysis (FIG. 6B) of that mRNA expression data.

FIGS. 7A-7C are graphs showing the simultaneous delivery of three distinct mRNAs: mRNA encoding firefly luciferase (FIG. 7A), GFP (FIG. 7B), and mCherry (FIG. 7C).

FIGS. 8A and 8B provide examples of amino acid (FIG. 8A; SEQ ID NO: 1) and mRNA (FIG. 8B; SEQ ID NO: 2) sequences for human PDX1 protein.

FIGS. 9A and 9B provide examples of amino acid (FIG. 9A; SEQ ID NO: 3) and mRNA (FIG. 9B; SEQ ID NO: 4) sequences for human MAFA protein.

FIGS. 10A and 10B provide examples of amino acid (FIG. 10A; SEQ ID NO: 5) and mRNA (FIG. 10B; SEQ ID NO: 6) sequences for human NGN3 protein.

FIG. 11 is a bar graph showing the reduction of luciferase mRNA delivery to the pancreas when beta cells are depleted with streptozotocin (STZ).

FIG. 12 is a bar graph showing the total luminescence in the lungs, liver, spleen, and pancreas after intraperitoneal delivery of lipid nanoparticles. The lipid nanoparticles were prepared using 306Oᵢ₁₀ as the ionizable lipidoid with either DOTAP, 18:1 EPC, or DDAB as the cationic helper lipid.

FIG. 13 provides an example mRNA sequence for human hepatocyte growth factor (HGF), transcript variant 1 (SEQ ID NO: 7).

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values.

As used herein, “a” and “an” refer to one or more.

The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” those stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

“Treatment” in the context of a disease or disorder, a marker for a disease or a disorder, or a symptom of a disease or disorder, can refer to a clinically-relevant and/or a statistically significant decrease or increase in an ascertained value for a clinically-relevant marker from outside a normal range towards, or to, a normal range. The decrease or increase can be, for example, at least 10%, at least 20%, at least 30%, at least 40%, or more, to a level accepted as either a therapeutic goal, or a level within the range of normal for an individual without such disease or disorder, or, in the case of a lowering of a value, to below the level of detection of an assay. The decrease or increase can be to a level accepted as within the range of normal for an individual without such disease or disorder, which can also be referred to as a normalization of a level. The reduction or increase can be the normalization of the level of a sign or symptom of a disease or disorder, that is, a reduction in the difference between the subject level of a sign of the disease or disorder and the normal level of the sign for the disease or disorder (e.g., to the upper level of normal when the value for the subject must be decreased to reach a normal value, and to the lower level of normal when the value for the subject must be increased to reach a normal level).

The compositions described herein may include as an active agent any anionic active agent, including, without limitation, a nucleic acid reagent, such as, without limitation, a DNA, an RNA (e.g., an mRNA), an antisense reagent, or an RNAi (RNA interference) reagent, or a negatively-charged polypeptide.

As used herein, the terms “cell” and “cells” refer to any types of cells from any animal, such as, without limitation, rat, mouse, monkey, and human. For example and without limitation, cells can be progenitor cells, e.g., pluripotent cells, including stem cells, induced pluripotent stem cells, multipotent cells, or differentiated cells, such as endothelial cells and smooth muscle cells. “Cells” may be in vivo, e.g., as part of a tissue or organ, or in vitro, such as a population of cells, such as, for example, a population of cells enriched for a specific cell type, such as, without limitation, a progenitor cell or a stem cell.

A composition is “biocompatible” in that the composition and, where applicable, elements thereof, or degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage.

“Therapeutically effective amount,” as used herein, can include the amount of an lipid-containing particle, such as an LNP, as described herein that, when administered to a subject having a disease, can be sufficient to effect treatment of the disease (e.g., by diminishing, ameliorating, or maintaining the existing disease or one or more symptoms of disease). The “therapeutically effective amount” may vary depending on the lipid-containing particle, such as an LNP, how the composition is administered, the ultrasound treatment protocol, the disease and its severity and the history, age, weight, family history, genetic makeup, the types of preceding or concomitant treatments, if any, and other individual characteristics of the subject to be treated.

A “therapeutically-effective amount” can also include an amount of an agent that produces a local or systemic effect at a reasonable benefit/risk ratio applicable to any treatment. Lipid-containing particle, such as an LNP, employed in the methods described herein may be administered in a sufficient amount to produce a reasonable benefit/risk ratio applicable to such treatment.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting a therapeutic agent for delivery to a patient. Each carrier can be “acceptable” in the sense of being compatible with the other ingredients of the formulation and not injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; and (24) other non-toxic compatible substances employed in pharmaceutical formulations.

A “group” or “functional group” is a portion of a larger molecule comprising or consisting of a grouping of atoms and/or bonds that confer a chemical or physical quality to a molecule. A “residue” is the portion of a compound or monomer that remains in a larger molecule, such as a polymer chain, after incorporation of that compound or monomer into the larger molecule. A “moiety” is a portion of a molecule, and can comprise one or more functional groups, and in the case of an “active moiety” can be a characteristic portion of a molecule or compound that imparts activity, such as pharmacological or physiological activity, to a molecule as contrasted to inactive portions of a molecule such as esters of active moieties, or salts of active agents.

As used herein, the term “polymer composition” is a composition comprising one or more polymers. As a class, “polymers” includes, without limitation, homopolymers, heteropolymers, copolymers, block polymers, block co-polymers and can be both natural and synthetic. Homopolymers contain one type of building block, or monomer, whereas copolymers contain more than one type of monomer.

A polymer “comprises” or is “derived from” a stated monomer if that monomer is incorporated into the polymer. Thus, the incorporated monomer that the polymer comprises is not the same as the monomer prior to incorporation into the polymer, in that at the very least, during incorporation of the monomer, certain groups, e.g., terminal groups, that are modified during polymerization are changed, removed, and/or relocated, and certain bonds may be added, removed, and/or modified. An incorporated monomer is referred to as a “residue” of that monomer. A polymer is said to comprise a specific type of linkage if that linkage is present in the polymer. Unless otherwise specified, molecular weight for polymer compositions refers to weight average molecular weight (Mw). A “moiety” can include a residue or group of residues within a larger polymer.

As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. “Lower alkyl” refers to C₁-C₆ alkyl. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, n-butyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Unsaturated alkyl” may comprise one or more, e.g., 1, 2, 3, 4, or 5, carbon-to-carbon double bonds and alternatively may be referred to as alkene or alkenyl, as described below. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

“Alkene or alkenyl” can include straight, branched chain, or cyclic hydrocarbyl groups including, e.g., from 2 to about 20 carbon atoms, such as, without limitation C₆₋₂₄ groups in the case of fatty acids, having one or more, e.g., 1, 2, 3, 4, or 5, carbon-to-carbon double bonds, and may be referred to as “unsaturated alkyl” in the context of fatty acids an lipids. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethenylene (—CH═CH—) and all stereoisomeric and conformational isomeric forms thereof. “Substituted alkenylene” can refer to divalent substituted alkene. “Optionally substituted alkenylene” can refer to alkenylene or substituted alkenylene.

An “ester” is represented by the formula —OC(O)R, where R can be an alkyl, alkenyl, or group described above.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne, and 4-octyne. An alkynyl group can be unsubstituted or optionally substituted with one or more substituents as described herein below. An alkyne or alkynyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkynyl group can be any alkynyl group substituted with any number of halogen atoms. The term “alkynylene” refers to divalent alkyne. Examples of alkynylene include without limitation, ethynylene, propynylene. “Substituted alkynylene” refers to divalent substituted alkyne.

“PEG” refers to polyethylene glycol. “PEGylated” refers to a compound comprising a moiety, comprising two or more consecutive ethylene glycol moieties. Non-limiting examples of PEG moieties for PEGylation of a compound include, one or more blocks of from 1 to 200 ethylene glycol units, such as —(O—CH₂—CH₂)ₙ—, —(CH₂—CH₂—O)ₙ—, or —(O—CH₂—CH₂)ₙ—OH, where n ranges, for example and without limitation, from 1 to 200 or from 1 to 100, for example from 1 to 5, or 1.

“Aryl,” alone or in combination refers to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” refers to divalent substituted aryl. “Optionally substituted arylene” refers to arylene or substituted arylene. A “polycyclic aryl group” and related terms, such as “polycyclic aromatic group” refers to a group composed of at least two fused aromatic rings. “Heteroaryl” or “hetero-substituted aryl” refers to an aryl group substituted with one or more heteroatoms, such as N, O, P, and/or S. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

“Cycloalkyl” refers to monocyclic, bicyclic, tricyclic, or polycyclic, 3- to 14-membered ring systems, which are either saturated, or partially unsaturated. The cycloalkyl group may be attached via any atom. Cycloalkyl also contemplates fused rings wherein the cycloalkyl is fused to an aryl or hetroaryl ring. Representative examples of cycloalkyl include, but are not limited to, cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with at least 1, 2, or 3 substituents, attached at any available atom to produce a stable compound, wherein the substituents are as described herein.

“Carboxyl” or “carboxylic” refers to group having an indicated number of carbon atoms, where indicated, and terminating in a —C(O)OH group, thus, having the structure —R—C(O)OH, where R is an unsubstituted or substituted divalent organic group that can include linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc. “Amine” or “amino” refers to group having the indicated number of carbon atoms, where indicated, and terminating in a —NH₂ group, thus, having the structure —R—NH₂, where R is a unsubstituted or substituted divalent organic group that, e.g., includes linear, branched, or cyclic hydrocarbons, and optionally comprises one or more heteroatoms. The term “alkylamino” refers to a radical of the formula —NHR^{x} or —NR^{x}R^{x} where each R^{x} is, independently, an alkyl radical as defined above.

Terms combining the foregoing refer to any suitable combination of the foregoing, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkenylheteroarylalkyl, alkylheteroarylalkenyl, alkylheteroarylalkynyl, alkynylheteroarylalkyl, alkenylheteroarylalkenyl, alkenylheteroarylalkynyl, alkylheterocyclylalkyl, alkynylheteroarylalkenyl, alkynylheteroarylalkynyl, alkylheterocyclylalkenyl, alkylhererocyclylalkynyl, alkenylheterocyclylalkyl, alkenylheterocyclylalkenyl, alkynylheterocyclylalkyl, alkenylheterocyclylalkynyl, alkynylheterocyclylalkenyl, alkylaryl, alkynylheterocyclylalkynyl, alkenylaryl, alkynylaryl, alkylheteroaryl, alkenylheteroaryl, alkynylhereroaryl. As an example, “arylalkylene” refers to a divalent alkylene wherein one or more hydrogen atoms in an alkylene group is replaced by an aryl group, such as a (C₃-C₈)aryl group. Examples of (C₃-C₈)aryl-(C₁-C₆)alkylene groups include without limitation 1-phenylbutylene, phenyl-2-butylene, 1-phenyl-2-methylpropylene, phenylmethylene, phenylpropylene, and naphthylethylene. The term “(C₃-C₈)cycloalkyl-(C₁-C₆)alkylene” refers to a divalent alkylene wherein one or more hydrogen atoms in the C₁-C₆ alkylene group is replaced by a (C₃-C₈)cycloalkyl group. Examples of (C₃-C₈)cycloalkyl-(C₁-C₆)alkylene groups include without limitation 1-cycloproylbutylene, cycloproyl-2-butylene, cyclopentyl-1-phenyl-2-methylpropylene, cyclobutylmethylene, and cyclohexylpropylene.

A fatty acid is an aliphatic monocarboxylic acid, comprising a carboxyl group linked to an aliphatic hydrocarbyl group which may be saturated or unsaturated. A hydrocarbyl or hydrocarbon group refers to a group of carbon and hydrogen atoms, such as alkyl, alkenyl (alternatively, unsaturated alkyl), or aryl groups. By “aliphatic”, it is meant acyclic or cyclic, saturated or unsaturated hydrocarbon compounds, excluding aromatic compounds. The aliphatic group of fatty acids is typically a linear chain of carbons, but fatty acids and substituted fatty acids as a class include linear, branched, and/or cyclic carbon chains. As used herein, fatty acids include both natural and synthetic aliphatic carboxylic acids. Fatty acids can have an aliphatic chain of from three to 40 carbon atoms (for example, as used herein, “a (C3-C40) fatty acid”). Hydrogen atoms of a compound, such as a fatty acid may be substituted with a group or moiety (hereinafter referred to as a “substituent”), to produce a substituted fatty acid. Fatty acids and substituted fatty acids may be referred to as “optionally substituted fatty acids”) Fatty acids, and fatty acid groups, may be referred to by the number of carbon atoms and the number of double bonds, e.g., C10:0, referring to a fatty acid or fatty acid group having 10 carbon atoms and zero double bonds. Likewise, C18:1 refers to a fatty acid with an 18-carbon chain having one double bond, such as oleic acid.

Unsaturated fatty acids and substituted unsaturated fatty acids (collectively “optionally substituted unsaturated fatty acids”) comprise one or more carbon-carbon double bonds, or an alkenyl group (e.g., vinyl group) in their aliphatic chains. The individual carbon atoms of the alkenyl group are referred to herein as alkenyl carbons. Unless specified, any carbon-carbon double bond in the alkyl chain of the described optionally substituted unsaturated fatty acids independently may be E (trans) or Z (cis) geometric isomers, or mixtures thereof.

Fatty acids may include, without limitation: C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39, and C40 fatty acids. The fatty acids may be saturated (zero double bonds), or unsaturated, e.g., with 0, 1, 2, 3, 4, 5, 6, or more double bonds. Non-limiting examples of saturated fatty acids include: propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, and tetracontylic acid. Non-limiting examples of unsaturated fatty acids include: crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-y-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, and herring acid. Compounds described herein, including fatty acids and substituted fatty acids can exist in various isomeric forms, including configurational, geometric, and conformational isomers, as well as existing in various tautomeric forms, such as those that differ in the point of attachment of a hydrogen atom. The term “isomer” is intended to encompass all isomeric forms of a compound of this invention, including tautomeric forms of the compound.

Certain compounds described here may have asymmetric centers and therefore exist in different enantiomeric and diastereomeric forms. A compound can be in the form of an optical isomer or a diastereomer. Accordingly, compounds described herein include their optical isomers, diastereoisomers and mixtures thereof, including a racemic mixture unless otherwise specified. Optical isomers of the compounds of the invention can be obtained by known techniques such as asymmetric synthesis, chiral chromatography, and simulated moving bed technology, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

Lipids, as a group, includes glycerides and phospholipids. A “glyceride” is an ester of glycerol (propane 1,2,3-triol) with a fatty acid or a substituted fatty acid. Phospholipids are lipids containing phosphoric acid as mono- or di-esters, such as phosphatidic acids and phosphoglycerides. Phosphoglycerides are di-esters of glycerol, which are glycerol derivatives in which one hydroxyl group of the glycerol is phosphodiester-linked to a group, such as a functional group, such as, for example and without limitation, a 2-amino ethanol or a choline (e.g., —O—CH₂—CH₂—N⁺(CH₃)₃) groups. A phosphatidylcholine is a phosphoglyceride with a choline linked to the glycerol moiety by a phosphodiester linkage. A glycerol-phosphoethanolamine is a phosphoglyceride with a 2-amino ethane group (e.g., —CH₂—CH₂—NH₃) linked to the glycerol moiety by a phosphodiester linkage. Amphipathic refers to a molecule or compound having both hydrophobic and hydrophilic parts, e.g., under physiological conditions.

As used herein, “cationic helper lipids” are positively charged amphiphilic lipids or glycerides that include a positively charged hydrophilic head group, a hydrophobic domain, and a linker bond that tethers the cationic hydrophobic head group and the hydrophobic domain. The cationic helper lipids remain positively charged under physiological conditions, such as at physiological pH. Non-limiting examples of cationic helper lipids include: 1,2-di-O-octadecenyl-3-trimethylammonium propane (DOTMA); glycero-ethylphosphocholine (EPC) lipids, such as 12:0-sn-glycero-3-ethylphosphocholine (12:0 EPC), 14:0 EPC, 16:0 EPC, 18:0 EPC, 18:1 EPC, 16:0-18:1 EPC, and 14:1-EPC; Dimethyldioctadecylammonium (DDAB); trimethylammonium-propane (TAP) lipids, such as 14:0 TAP, 18:0 TAP, or 18:1 TAP (DOTAP); 3β-[N-(N′,N′-dimethylaminoethane)-carbamoyl]cholesterol hydrochloride (DC-chol); N⁴-cholesteryl-spermine (GL67); 1,2-dioleyloxy-3-dimethylaminopropane (DODMA); 1,2-dioleoyl-3-dimethylammonium-propane (DODAP); and N1-[2-((1S)-1-[(3-aminopropyl)amino]-4-[di(3-amino-propyl)amino] butylcarboxamido)ethyl]-3,4-di[oleyloxy]-benzamide (MVL5), which are typically provided as salts thereof, such as chloride, bromide, trifluoromethanesulfonimide, or other salts. Exemplary cationic helper lipids are commercially available from Avanti Polar Lipids of Alabaster Alabama.

Lipid particles are provided. Examples of lipid-containing particles, as described herein comprise, without limitation:
a cationic helper lipid, such as positively-charged amphipathic lipid or glyceride, such as DOTMA, an EPC, DDAB, a TAP, DC-Chol, GL67, DODMA, DOTAP, DODAP, MVL5, or other molecules with a hydrophobic component or tails attached to a net positively charged moiety or moieties;
cholesterol or a derivative thereof;
a PEG-based compound, such as a PEG-containing polymer or PEGylated fatty acid-containing compound; and
an ionizable lipidoid, forming a cation at an acidic pH, e.g., less than 6.5.

The lipid-containing particles may be described as lipid nanoparticles or lipid microparticles, depending on their size. The particles may be used to deliver any compatible cargo or active agent, such as, without limitation, a polynucleotide, a drug, a protein or peptide, a small molecule, or a gas. The particles may be used to deliver an anionic or polyanionic cargo to the pancreas. The anionic or polyanionic cargo may be a nucleic acid, such as, without limitation: an mRNA, an antisense reagent, an RNAi agent, a genetic vector or recombinant construct such as a plasmid or other extrachromosomal or chromosome-targeting nucleic acid, a nucleic acid for use in gene editing, a recombinant or natural viral genome, DNA comprising a gene, a ribozyme, or an aptamer. For example and without limitation, the agent or cargo may be an RNA (e.g., mRNA, RNAi, dsRNA, siRNA, shRNA, miRNA, an antisense RNA, a guide RNA (gNA), long non-coding RNAs (IncRNA), a base editing gRNA (beRNA), a prime editing gRNA (pegRNA), or a transfer RNA (tRNA)). The cargo may be an mRNA, e.g., a capped and optionally PEGylated mRNA, encoding a therapeutic polypeptide or protein, or an immunogen. In one example, the mRNA encodes: pancreatic and duodenal homeobox 1 (PDX1), MafA, and/or neurogenin-3 (Ngn3) mRNAs. By “encoding”, it is meant that a nucleic acid, such as an mRNA or DNA comprises a coding sequence (CDS, alternatively referred to as an open reading frame or ORF) that may be translated to produce a specified protein. The nucleic acid, when an mRNA, comprises not only the CDS, but any sequences required for translation of the CDS in a target cell or expression system, such as: a 5′ cap, 5′ and 3′ untranslated regions, start and stop codons, and polyA tails in the case of mRNA, as are broadly-known in the art. The nucleic acid, when a DNA, may comprise a gene for encoding a stated protein, and comprises any suitable transcription control elements and a CDS for transcription of an mRNA encoding the specified protein, as are broadly-known in the art. The DNA, along with the gene, may comprise suitable vector sequences, such as viral sequences, or other sequences, that facilitate extrachromosomal expression of the gene, or chromosomal integration of the gene, such as adenovirus, adeno-associated virus, herpesvirus, or retrovirus sequences.

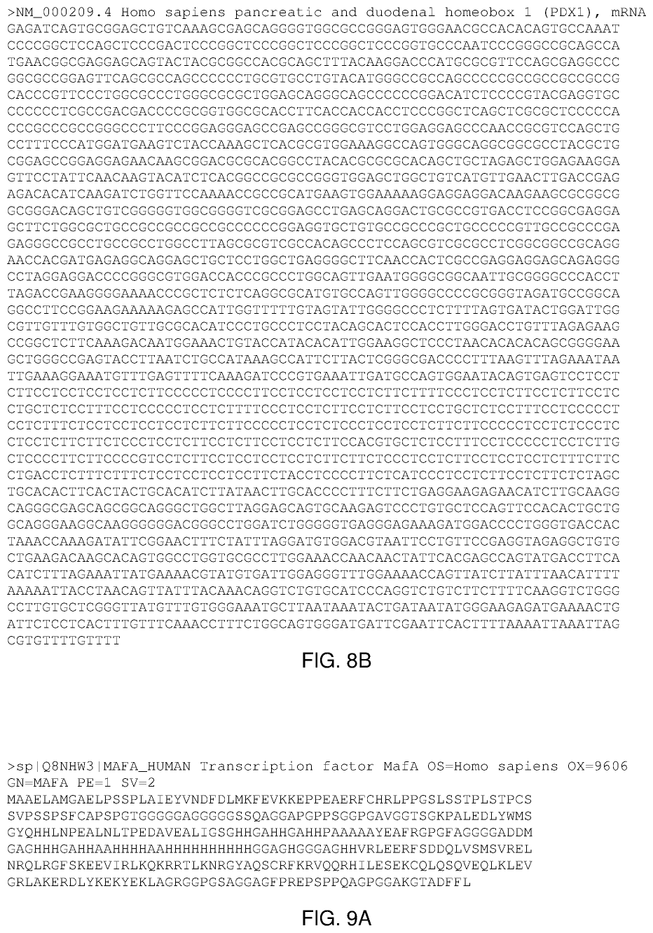

PDX1 is a transcription factor, referring to Pancreas/duodenum homeobox protein 1 (gene, PDX1). An example of an amino acid sequence for human PDX1 is provided in FIG. 8A (see, e.g., UniProtKB—P52945 (PDX1_HUMAN) and NCBI Reference Sequence: NP_000200.1; SEQ ID NO: 1). An exemplary mRNA sequence encoding human PDX1 is provided in FIG. 8B (see, e.g., NCBI Reference Sequence: NM_000209.4; SEQ ID NO: 2).

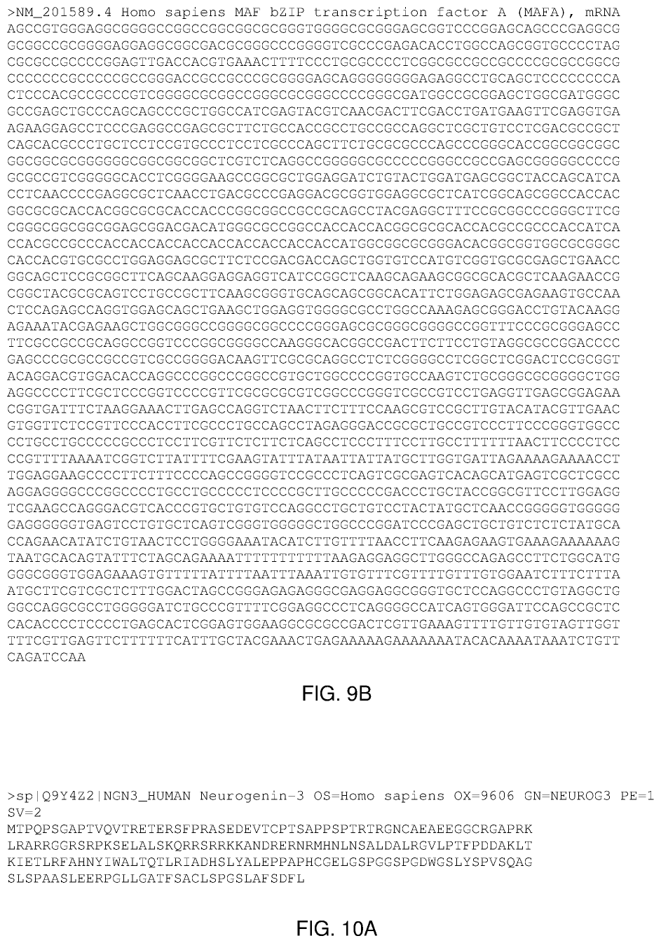

MafA is a transcription factor, referring to MAF bZIP transcription factor A (gene, MAFA). An exemplary amino acid sequence for human MafA is provided in FIG. 9A (see, e.g., UniProtKB—Q8NHW3 (MAFA_HUMAN) and NCBI Reference Sequence: NP_963883.2; SEQ ID NO: 3). An exemplary mRNA sequence encoding human MafA is provided in FIG. 9B (see, e.g., NCBI Reference Sequence: NM_201589.4; SEQ ID NO: 4).

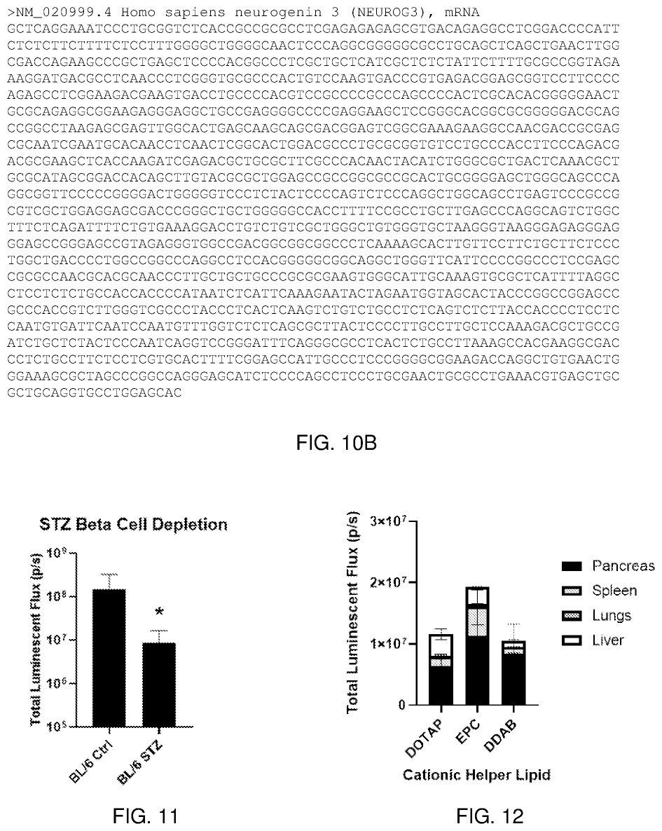

Ngn3 is a transcription regulator, and refers to Neurogenin-3 (gene, NEUROG3). An exemplary amino acid sequence for human Ngn3 is provided in FIG. 10A (see, e.g., UniProtKB—Q9Y4Z2 (NGN3_HUMAN) and NCBI Reference Sequence: NP_066279.2; SEQ ID NO: 5). An exemplary mRNA sequence encoding human MafA is provided in FIG. 10B (see, e.g., NCBI Reference Sequence: NM_020999.4; SEQ ID NO: 6).

mRNA sequences encoding a defined polypeptide may differ in their ORF sequences due to codon degeneracy, such that two mRNAs encoding the same polypeptide can have sequence identities of 70% or less, although certain codons may be favored in any given organism. A “codon-optimized” nucleic acid refers to a nucleic acid sequence that has been altered such that the codons are optimal for expression in a particular system (such as a particular organism, species, or group of species). For example, a nucleic acid sequence can be optimized for expression in mammalian cells. Codon optimization does not alter the amino acid sequence of the encoded protein. Codon optimized constructs, e.g., plasmids, may be designed and produced by any suitable method, and constructs may be tested for their expression in any applicable recombinant protein production system.

A conservative substitution is a substitution of one amino acid residue in a protein sequence for a different amino acid residue having similar biochemical properties. Typically, conservative substitutions have little to no impact on the activity of a resulting polypeptide. For example, ideally, a PDX1, MafA, or NGN3 protein including one or more conservative substitutions (for example 1, 2, 3, 4, 5, 6, 7, 8, 9, or 10 substitutions) retains the structure and function of the wild-type protein, namely, in the context of the present disclosure, the ability to elicit a cellular response, such as, for example and without limitation, stimulation of pancreatic acinar or alpha cells to induce their conversion to insulin-producing beta cells, in the case of delivery of PDX1-, MafA-, and/or NGN3-encoding mRNAs. A polypeptide can be produced to contain one or more conservative substitutions by manipulating the nucleotide sequence that encodes that polypeptide using, for example, standard procedures such as site-directed mutagenesis or PCR. Conservative amino acid substitutions may be determined, for example, by comparing intra- or inter-species variations in amino acid sequences of a specified protein, by computer modeling or polypeptides, or through use of substitution matrices. Signal peptides, cleavable peptide tags, or other useful sequences to the may be attached C-terminus or N-terminus of a polypeptide, e.g., by encoding in-frame.

“Sequence identity” refers to the similarity between nucleic acid or amino acid sequences is expressed in terms of the similarity between the sequences, otherwise referred to as sequence identity. Sequence identity may be measured in terms of percentage identity (or similarity or homology); the higher the percentage, the more similar the two sequences are. Homologs, orthologs, or variants of a polypeptide will possess a relatively high degree of sequence identity when aligned using standard methods. Methods of alignment of sequences for comparison are well-known in the art, including various programs and alignment algorithms.

Once aligned, the number of matches may be determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide are typically characterized by possession of at least about 75%, for example, at least about 80%, 85%, 90%, 91%, 92%, 93%, 94%, 95%, 96%, 97%, 98%, or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85%, at least 90%, or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman (Smith et al., “Comparison of biosequences”, Adv. Appl. Math. 1981, 2:482), by the homology alignment algorithm of Needleman & Wunsch, (Needleman, et al., “A General Method Applicable to the Search for Similarities in the Amino Acid Sequence of Two Proteins”, J. Mol. Biol., 1970, 48:443-453), by the search for similarity method of Pearson & Lipman (Pearson, et al., “Improved tools for biological sequence comparison”, Proc. Natl. Acad. Sci. USA, 1988, 85:2444-2448), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. One example of a useful algorithm is PILEUP. PILEUP uses a simplification of the progressive alignment method of Feng & Doolittle (Feng, G, et al., “Progressive Sequence Alignment as a Prerequisite to Correct Phylogenetic Trees”, J Mol Evol, 1987, 25:351-360). The method used is similar to the method described by Higgins & Sharp (Higgins et al. “Fast and sensitive multiple sequence alignments on a microcomputer”, Bioinformatics, 1989, 5:151-153). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0.

Another example of algorithms that are suitable for determining percent sequence identity and sequence similarity are the BLAST and the BLAST 2.0 algorithm, which are described in Altschul, et al., “Basic Local Alignment Search Tool”, J. Mol. Biol., 1990, 215:403-410) and Altschul, et al., “Gapped BLAST and PSI-BLAST: a new generation of protein database search programs”, Nucleic Acids Research, 1997, 25(17):3389-3402. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see, Henikoff, et al., “Amino acid substitution matrices from protein blocks”, Proc. Natl. Acad. Sci. USA, 1992, 89:10915-10919). An oligonucleotide is a linear polynucleotide sequence of up to about 100 nucleotide bases in length.

As used herein, reference to “at least 80% identity” (or similar language) refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

The lipid particles described herein may also be incorporated into drug delivery devices, e.g., drug products, dosage forms, unit dosage forms, etc. The lipid particles may be used to encapsulate agents including polynucleotides, small molecules, proteins, peptides, metals, organometallic compounds, etc.

“Nucleic acids” include DNA and RNA as is found naturally, and chemically-modified nucleic acids, as are broadly-known, may find use in the present compositions and methods. Nucleic acids useful in the compositions and methods described herein may be polyanionic nucleic acids, having an overall negative charge under neutral or physiological conditions, such as in an aqueous solution pH 6-8, e.g., in water, blood, serum, Ringer's, or normal saline. A nucleic acid may comprises a phosphorus-containing moiety, such as a phosphate and/or a phosphorothioate moiety, and, therefore, would be polyanionic. Non-limiting examples of nucleic acids include RNAi agents, antisense reagents, aptamers, and ribozymes, among others (see, e.g., Bajan S, Hutvagner G. “RNA-Based Therapeutics: From Antisense Oligonucleotides to miRNAs”, Cells, 2020, 9(1):137; Invitrogen RNAi Handbook, ThermoFisher Scientific 2015; and Kilanowska, A., et al., “In vivo and in vitro studies of antisense oligonucleotides—a review”, RSC Adv., 2020, 10, 34501). Nucleic acids may be unmodified (e.g., natural) or chemically-modified (see, e.g., Dar, S., et al., “siRNAmod: A database of experimentally validated chemically modified siRNAs”. Sci Rep, 2016, 6, 20031 and crdd.osdd.net/servers/sirnamod/).

Provided herein is a lipid-containing particle, e.g. a lipid nanoparticle or microparticle, formulation for parenteral or oral delivery for targeting the pancreas. The particles may be delivered intraperitoneally. The particles may be delivered intravenously, intramuscularly, subcutaneously, or intradermally. Suitable formulations for parenteral or oral delivery may be developed using standard drug formulation methodology. For parenteral delivery, the particles may be delivered in an aqueous solution, such as water, normal saline, phosphate-buffered saline, Ringer's, etc. Targeting the pancreas, in the context of the methods and compositions described herein, refers to the delivery of a therapeutic agent, such as a lipid particle reagent as described herein, intraperitoneally with the selective delivery of the therapeutic agent, e.g., the therapeutic cargo of the lipid particle, to the pancreas, to the exclusion of all, or most other major organs of the abdominal cavity, such as delivery to the pancreas with comparatively lower delivery to the lungs, liver, and spleen, or comparatively lower delivery to the lungs and liver. Lipid nanoparticles that incorporate positively-charged helper lipids aid the nanoparticles in specifically targeting the pancreas. These positively-charged components could include positively-charged helper lipids such as DOTMA, an EPC, DDAB, a TAP, DC-Chol, GL67, DODMA, DODAP, DOTAP, or MVL5 or other molecules with a hydrophobic component or tails attached to a net positively-charged moiety or moieties. Typical lipid nanoparticle formulations use amphiphilic, net neutral lipids such as phosphatidylethanolamines and phosphatidylcholines.

The lipid particles may have a neutral net charge at pH 7.0. The net charge of the lipid particle may be measured by any useful method or assay. In one example, it is measured by zeta potential (ζ-potential). The zeta potential of the particle may range from +2 to −2 at neutral pH, e.g. approximately 0. ζ-potential may be measured by any useful method, for example and without limitation using Malvern ZetaSizer Nano (Malvern Instruments), or an equivalent thereof. Determining ζ-potential is one way of measuring surface charge, and may not be sufficiently sensitive in all instances for measuring net surface charge of the lipid particles described herein, other methods of determining surface charge may be employed, such as the TNS method, see below for details. The TNS method uses 2-(p-toluidinyl)naphthalene-6-sulphonic acid (TNS), which only binds to cationic lipids and is commonly used to determine lipid pKa (see, e.g., Uebbing L, et al., “Investigation of pH-Responsiveness inside Lipid Nanoparticles for Parenteral mRNA Application Using Small-Angle X-ray Scattering”, Langmuir, 2020, 36(44):13331-13341).

The diameter of the lipid-containing particles may range from 1 micrometer (μm) to 1,000 μm (microparticles). The diameter of the particles range may range from 1 μm to 100 μm, from 1 μm to 10 μm, from 10 μm to 100 μm, from 100 μm to 1,000 μm, or from 1 μm to 5 μm. The diameter of the lipid particles may range from between 1 nanometers (nm) to 1,000 nm (nanoparticles), from 1 nm to 100 nm, from 1 nm to 10 nm, from 10 nm to 100 nm, from 100 nm to 1,000 nm, from 20 nm to 2,000 nm, or from 1 nm to 5 nm. The diameter of the particles range from between 1 picometers (pm) to 1,000 pm, from 1 pm to 100 pm, from 1 pm to 10 pm, from 10 pm to 100 pm, from 100 pm to 1,000 pm, or from 1 pm to 5 pm.

The lipid particles may be prepared using any useful method. These include, but are not limited to, spray drying, single and double emulsion solvent evaporation, solvent extraction, phase separation, and simple and complex coacervation, among other methods. The method of preparing the particles may be the double emulsion process and spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix. Methods developed for making particles for delivery of encapsulated agents are amply described in the literature. In one example, the lipid-containing particles are prepared by microfluidics (see, e.g., Chen D, et al., “Rapid discovery of potent siRNA-containing lipid nanoparticles enabled by controlled microfluidic formulation”, J Am Chem Soc., 2012, 134(16):6948-51 and Cayabyab, C, et al., “mRNA Lipid Nanoparticles: Robust low-volume production for screening high-value nanoparticle materials,” 2018, Precision NanoSystems, Inc., describing methods of making lipid nanoparticles, including suitable ratios for various constituents). Briefly, appropriate amounts of the ionizable lipidoid, the cationic helper lipid, the cholesterol or cholesterol derivative and PEG-based material are mixed in an appropriate solvent, such as 90% ethanol and 10% 10 millimolar (mM) sodium citrate and mixed with an appropriate amount of the cargo, such as mRNA or siRNA in 10 mM sodium citrate at a weight ratio of mRNA or siRNA to the (ionizable lipidoid+cholesterol or cholesterol derivative+cationic helper lipid+PEG-based material) of, for example and without limitation of 1:5-1000, e.g., 1:20. The lipid particles may be formed in a microfluidics device or by rapid pipetting. Particles may be diluted in a suitable aqueous solvent, such as phosphate buffered saline (PBS), and optionally dialyzed against the same or a different aqueous solvent.

If the particles prepared by any of the above methods have a size range outside of the desired range, the particles can be sized, for example, using a sieve or filter. The particle may also be coated. The particle may be coated with a targeting agent.

The lipid-containing particles comprise cholesterol or a derivative thereof, such as 3β[N-(N′,N′-dimethylaminoethane)-carbamoyl]cholesterol (DC-cholesterol).

The lipid-containing particles comprise a PEG (poly(oxyethylene))-based material (PEGylated), such as a PEGylated fatty acid-containing compound or PEG-containing block copolymer, such as a poloxamer. Non-limiting examples of PEG-based materials include: PEG-ceramide, PEG-monoglycerides or diglycerides such as PEG-DMG, PEG-PE, PEG-sphingosine, PEG-sphingomyelin, poloxamer, or DSPE carboxy PEG, where DMG refers to dimyristoyl-glycerol, PE refers to phosphoethanolamine, and DSPE refers to distearoyl-glycero-phosphoethanolamine. For instance, in certain embodiments, the PEGylated fatty acid-containing compound is a PEG-glyceride, for example and without limitation a PEG-diglyceride, such as C₁₄-PEG₂₀₀₀ DMG (commercially available from Avanti Polar Lipids, Birmingham, AL), C₁₄-PEG₂₀₀₀, C₁₅-PEG₂₀₀₀, C₁₆-PEG₂₀₀₀, C₁₈-PEG₂₀₀₀, C₈-PEG₇₅₀ ceramide, C₁₆-PEG₇₅₀ ceramide, C₈-PEG₂₀₀₀ ceramide, C₁₄-PEG₂₀₀₀ ceramide, C₁₅-PEG₂₀₀₀ ceramide, C₁₆-PEG₂₀₀₀ ceramide, C₁₈-PEG₂₀₀₀ ceramide, C₈-PEG₅₀₀₀ ceramide, C₁₆-PEG₅₀₀₀ ceramide, C₁₄-PEG₂₀₀₀ PE, C₁₅-PEG₂₀₀₀ PE, C₁₆-PEG₂₀₀₀ PE, C₁₈-PEG₂₀₀₀ PE, C₁₄-PEG₃₅₀ PE, C₁₄-PEG₅₀₀₀ PE, or DSPE carboxy PEG, which are commercially available from Avanti Polar Lipids, and the PEG-containing block copolymer is poloxamer F-127, poloxamer F-68, or poloxamer L-64. A PEG-glyceride refers to a PEGylated ester of glycerol and one or more fatty acids. In the case of a diglyceride, the fatty moieties may be the same or different.

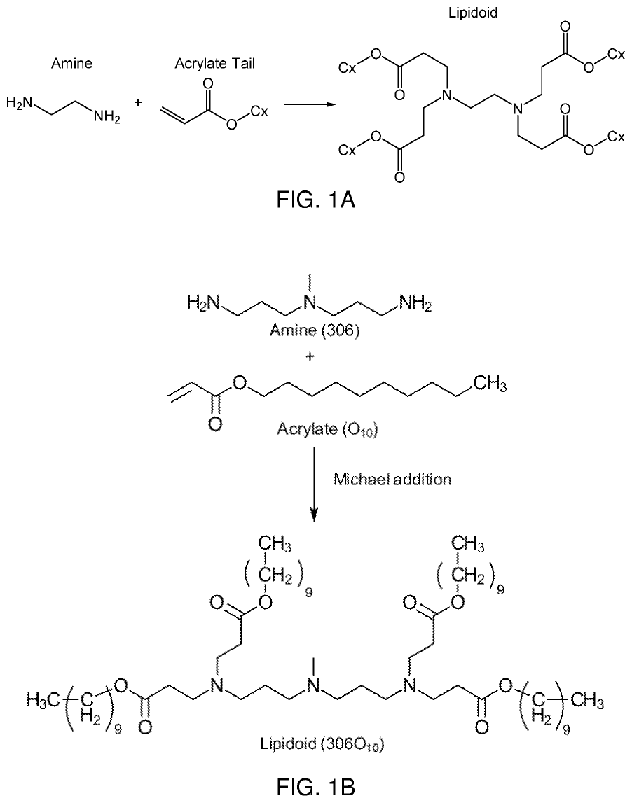

A lipidoid is a lipid-like molecule. An ionizable lipidoid is a lipidoid that forms an ion in acidic or basic conditions, such as a cation under acidic conditions. Non-limiting examples of ionizable lipidoids are provided in U.S. Pat. No. 9,439,968, generally forming lipidoids by conjugate addition of alkyl-acrylates to amines. FIG. 1 of that document is reproduced herein, and provides the general synthesis scheme of useful amino-lipidoids prepared from amines and alkyl-acrylates (see, FIGS. 1A and 1B). Also provided are useful amines, e.g., designated as 25, 32, 306, etc., and structures of alkyl-acrylates, e.g. O₁₀, O₁₁, O₁₂, O₁₃, and O₁₄. Lipidoids are designated in the examples below in reference to the amine and alkyl-acrylate used to make the ionizable lipidoid, e.g., 306O₁₀, referring to N,N-Bis(3-aminopropyl)methylamine conjugated to decyl acrylate.

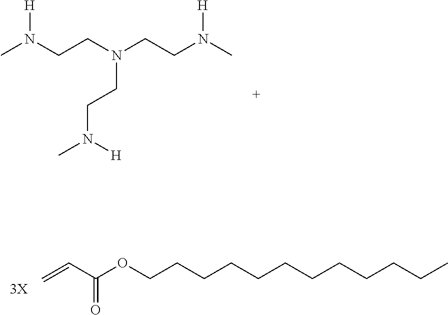

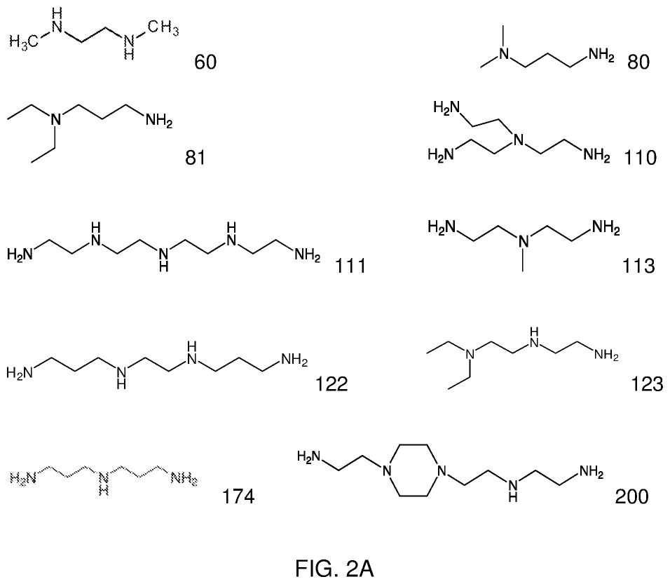

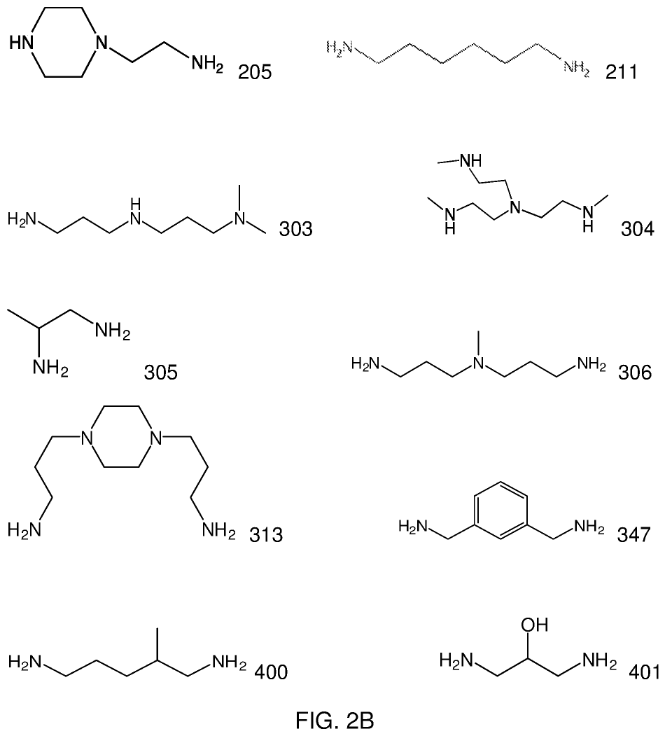

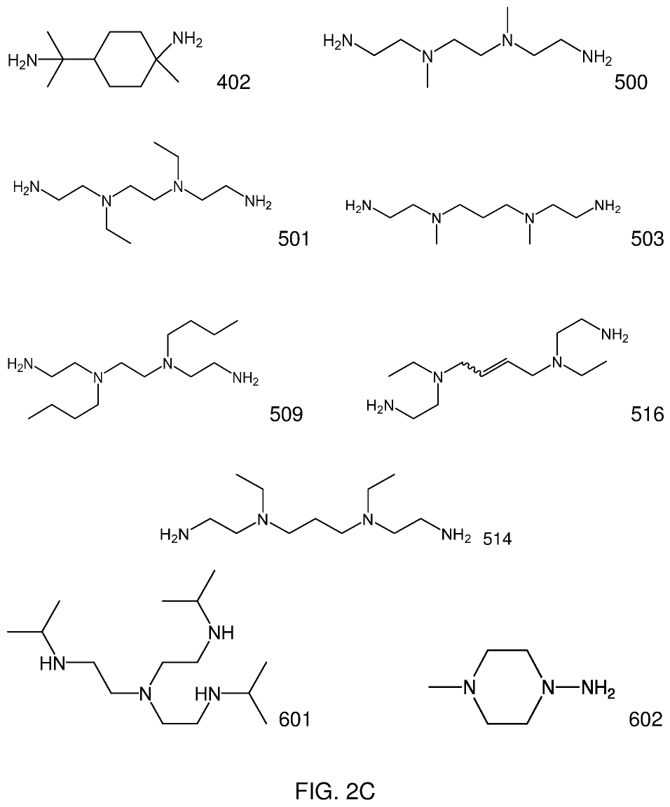

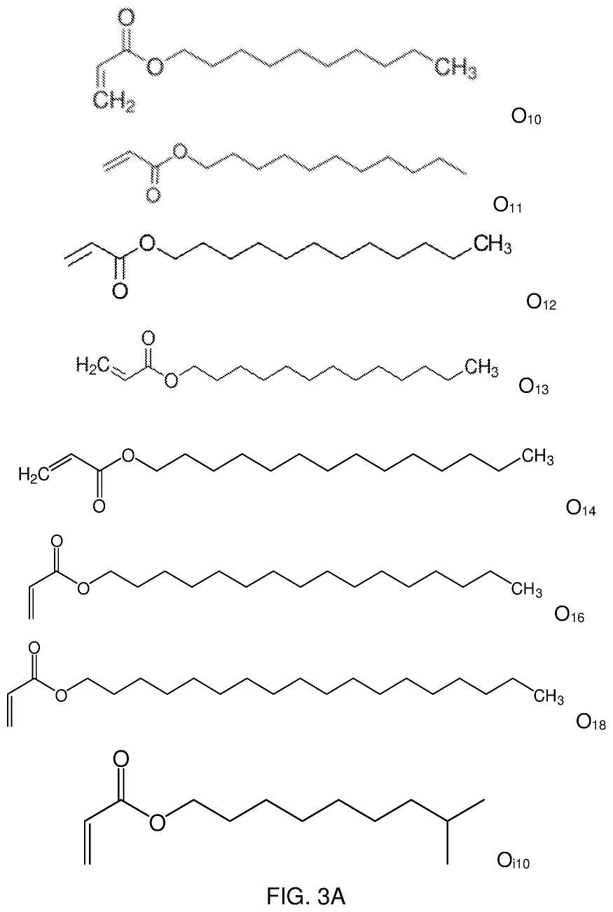

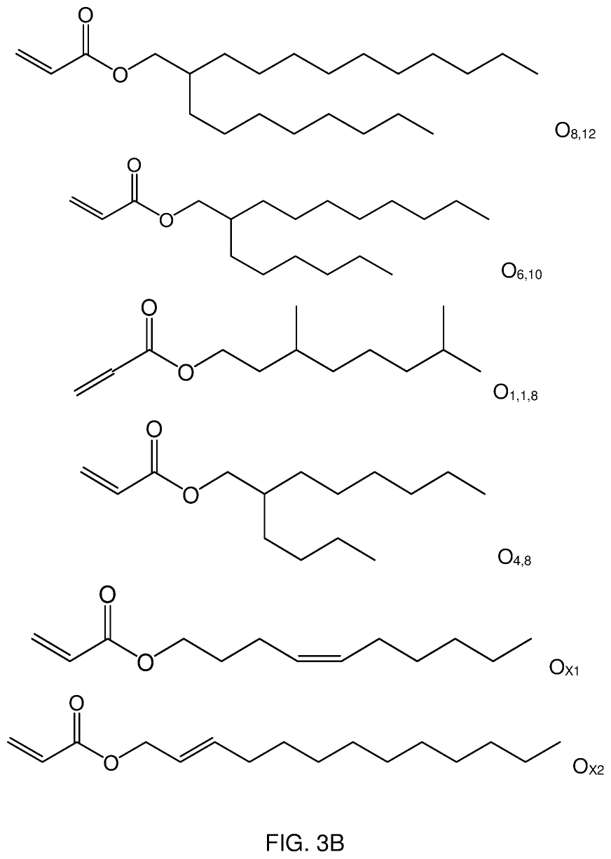

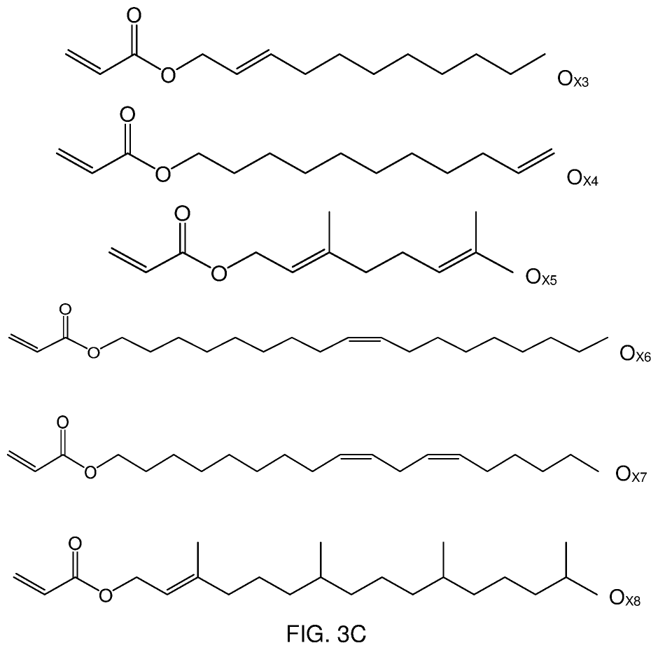

Lipidoids for preparation of LNPs for intraperitoneal delivery to the pancreas may include any combination, e.g., by Michael addition, of an alkylamine having from one to five amine moieties, and an alkyl or alkenyl acrylate. Lipidoids for preparation of LNPs for intraperitoneal delivery to the pancreas may include any combinatorial permutation of an amine depicted in FIGS. 2A-2C, and one or more acrylate depicted in FIGS. 3A-3C, e.g., as depicted in FIGS. 1A and 1B. Lipidoids for preparation of lipid-containing particles for intraperitoneal delivery to the pancreas may include any combinatorial permutation of an amine depicted in FIGS. 2A-2C, and one or more acrylate depicted in FIGS. 3A-3C. Additional examples of lipidoids are described in US Patent Application Publication No. 2020/0109113 A1, incorporated herein by reference for its description of additional exemplary lipidoid compounds, and uses therefor.

### EXAMPLES

### Example 1

### Materials and Methods

Lipid nanoparticle synthesis: The ionizable lipidoids 306Oᵢ₁₀, 200Oᵢ₁₀, and 514O_{6,10} were synthesized by reacting amine heads with acrylate tails using Michael addition chemistry. Lipidoids were combined with DOPE, cholesterol, and 1,2-dimyristoyl-sn-glycero-3-phosphoethanolamine-N-[methoxy(polyethylene glycol)-2000, ammonium salt (an exemplary PEGylated fatty acid-containing compound and a PEGylated phosphoethanolamine (PE), referred to in the following examples as C₁₄-PEG₂₀₀₀ PE) in a 35:16:46.5:2.5 molar ratio in 100% ethanol. To formulate mRNA lipid nanoparticles, the lipid solution was combined 1:1 volume/volume (vol/vol) ratio with a solution of mRNA encoding firefly luciferase (10:1 lipidoid:mRNA weight/weight (w/w) ratio) suspended in 10 mM sodium citrate (pH 4) by vortexing. Lipid nanoparticles were diluted 1:1 (vol/vol) in PBS (pH 7.4) and further dialyzed against PBS for one hour.

In vivo experiments: Lipid nanoparticles were delivered to C57BL/6 mice (6-8 weeks, female) at a dosage of 0.5 milligrams (mg) mRNA per kilogram (kg) body weight by intravenous (tail vein, IV) or intraperitoneal (IP) injection. Three hours later, mice were treated with D-luciferin for 15 minutes, then sacrificed and dissected. Organs were imaged for bioluminescence ex vivo using an IVIS (Perkin Elmer) imaging system. Data were analyzed using LivingImage software (Perkin Elmer) and plotted in GraphPad Prism.

### Results

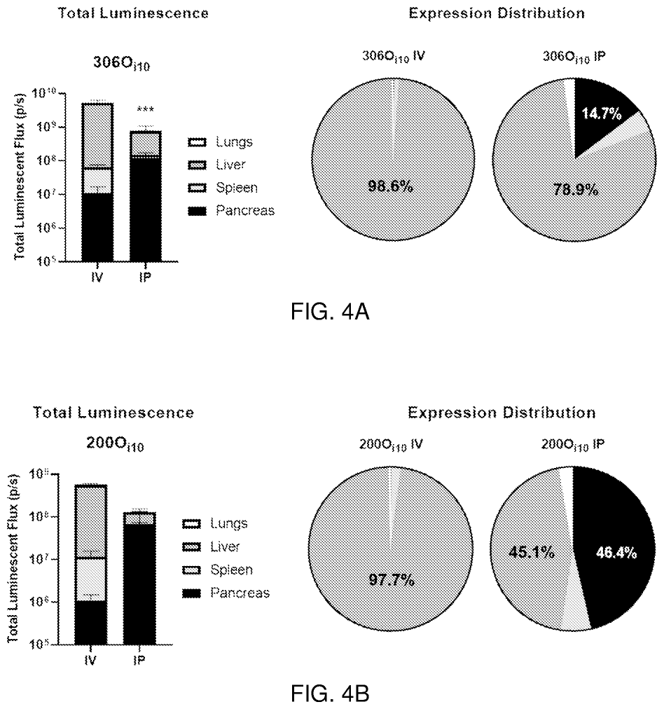

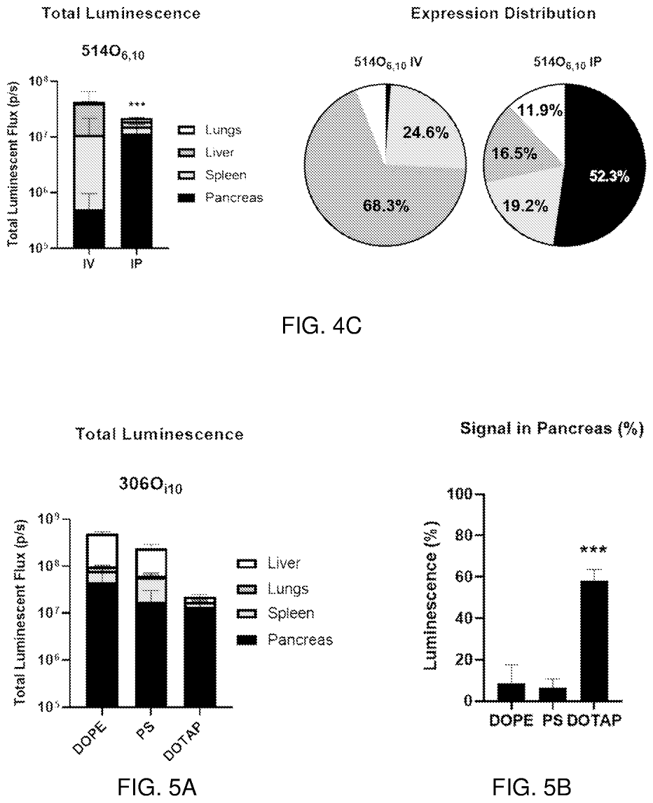

For all tested ionizable lipidoids (306Oᵢ₁₀, 200Oᵢ₁₀, and 514O_{6,10}), intraperitoneal injection significantly increased mRNA delivery to the pancreas relative to intravenous injection, as evidenced by an increase in bioluminescence signal indicating luciferase protein production (FIGS. 4A-4C). The results from these ionizable lipids show that intraperitoneal injection of lipid nanoparticles containing any ionizable lipid increase cargo delivery to the pancreas.

### Example 2

### Materials and Methods

Lipid nanoparticle synthesis: The ionizable lipidoids 306Oᵢ₁₀, 200Oᵢ₁₀, and 514O_{6,10} were synthesized by reacting amine heads with acrylate tails using Michael addition chemistry. The ionizable lipidoids were combined with one of three helper lipids (DOPE, PS, or DOTAP), cholesterol, and C₁₄-PEG₂₀₀₀ PE in a 35:40:22.5:2.5 molar ratio in 100% ethanol. To formulate mRNA lipid nanoparticles, the lipid solution was combined 1:1 (vol/vol) with a solution of mRNA encoding firefly luciferase (10:1 lipidoid:mRNA w/w) suspended in 10 mM sodium citrate (pH 4) by vortexing. Lipid nanoparticles were diluted 1:1 (vol/vol) in PBS (pH 7.4) and further dialyzed against PBS for one hour. In vivo experiments: The lipid nanoparticles were delivered to C57BL/6 mice (6-8 weeks, female) at a dosage of 0.5 mg mRNA/kg body weight by intravenous (tail vein) or intraperitoneal injection. Three hours later, mice were treated with D-luciferin for 15 min, then sacrificed and dissected. Organs were imaged for bioluminescence ex vivo using an IVIS imaging system. Data were analyzed using LivingImage software and plotted in GraphPad Prism.

### Results

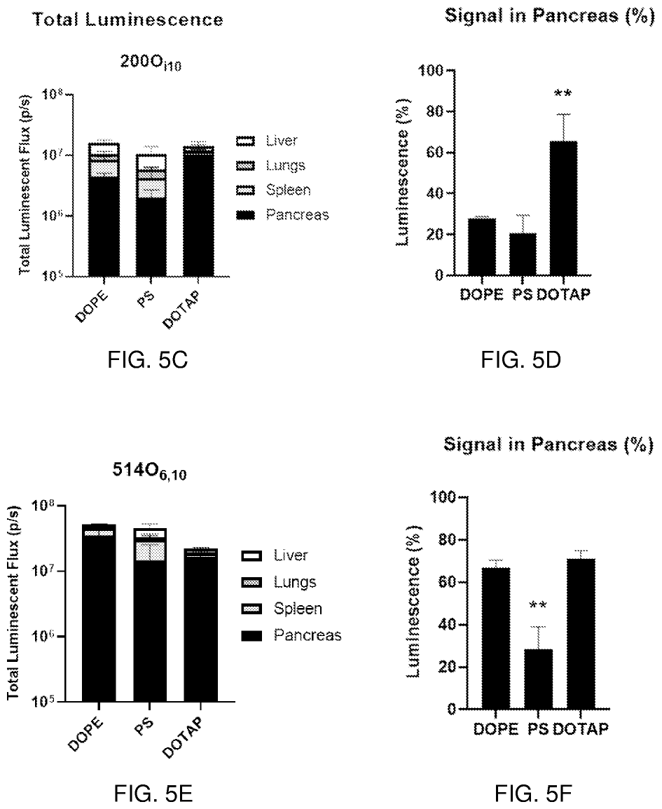

For all tested ionizable lipids (306Oᵢ₁₀, 200Oᵢ₁₀, and 514O_{6,10}), the cationic helper lipid DOTAP significantly increased the fraction of the total signal that occurs in the pancreas (except for 514O_{6,10}, for which DOPE also facilitated high pancreatic specificity) (FIGS. 5A-5F). The cationic helper lipid DOTAP reduces off-target mRNA delivery to the liver and spleen independently of the lipidoid component, thereby improving specificity for the pancreas. It was determined that the following ranges of molar ratios in Tables 1 and 2 performed best:

TABLE 1

| | | |
|---|---|---|
| Lower Limit | Component | Upper Limit |

| | | | | |
|---|---|---|---|---|
| 20 | ≤ | Ionizable Lipid | ≤ | 55 |
| 10 | ≤ | DOTAP | ≤ | 60 |
| 10 | ≤ | Cholesterol | ≤ | 50 |
| 1 | ≤ | C₁₄-PEG₂₀₀₀ PE | ≤ | 2.5 |

TABLE 2

| | | |
|---|---|---|
| Lower Limit | Component | Upper Limit |

| | | | | |
|---|---|---|---|---|
| 20 | ≤ | Ionizable Lipid | ≤ | 45 |
| 30 | ≤ | DOTAP | ≤ | 50 |
| 10 | ≤ | Cholesterol | ≤ | 46.5 |
| 1.25 | ≤ | C₁₄-PEG₂₀₀₀ PE | ≤ | 2.5 |

### Example 3

### Materials and Methods

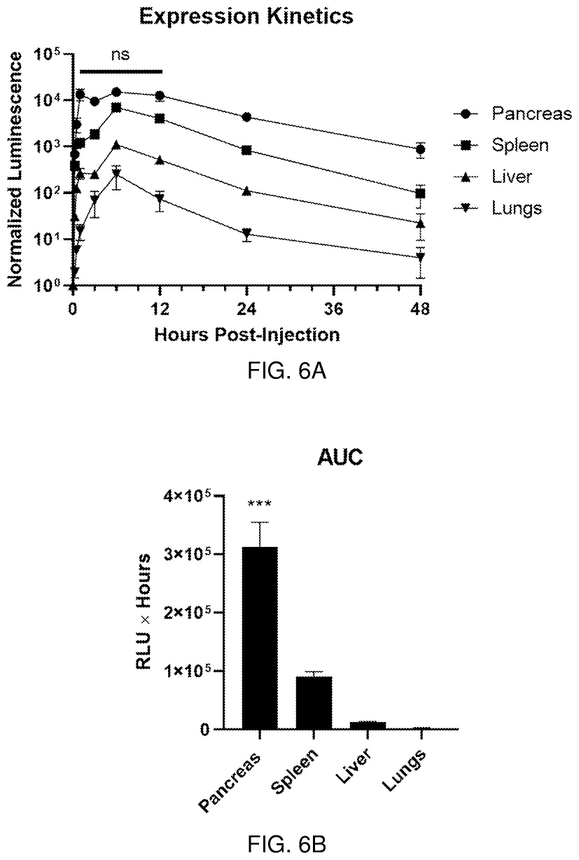

As shown in FIGS. 6A and 6B, protein expression induced using the strategy as described in Examples 1 and 2 persists for at least 48 hours and is significantly greater in the pancreas than in the liver, spleen, or lungs. The expression of firefly luciferase, induced by mRNA lipid nanoparticles, persists for at least 48 hours and is significantly greater in the pancreas than in the spleen, liver, or lungs, as determined by area under the curve calculations. Luminescence was determined at multiple time points as described in Example 1 and 2. The LNP comprised 35% 306Oᵢ₁₀, 40% DOTAP, 22.5% cholesterol, and 2.5% C₁₄-PEG₂₀₀₀ PE, and were synthesized as described in Example 2.

### Example 4

### Materials and Methods

Lipid nanoparticle synthesis: Three different mRNAs encoding three different fluorescent proteins were incorporated into lipid nanoparticles as described in Example 2 and expressed preferentially in the pancreas. The three mRNAs that encoded luciferase, green fluorescent protein (GFP), and mCherry fluorescent proteins, respectively, were incorporated into lipid nanoparticles comprising 35% 306Oᵢ₁₀, 40% DOTAP, 22.5% cholesterol, and 2.5% C₁₄-PEG₂₀₀₀ PE. To formulate the mRNA lipid nanoparticles, the lipid solution was combined 1:1 volume/volume (vol/vol) ratio with a solution of mRNA encoding firefly luciferase, mRNA encoding GFP, and mRNA encoding mCherry fluorescent proteins (10:1 lipidoid:mRNA weight/weight (w/w) ratio) suspended in 10 mM sodium citrate (pH 3 to 4) by vortexing. Lipid nanoparticles were diluted 1:1 (vol/vol) in PBS (pH 7.4) and further dialyzed against PBS for one hour.

In vivo experiments: Lipid nanoparticles were delivered to C57BL/6 mice (6-8 weeks, female) at a dosage of 1.0 mg mRNA/kg body (0.33 mg/kg each mRNA) weight by intraperitoneal injection. Six hours later, mice were intraperitoneally injected with D-luciferin. Fifteen minutes later, the mice were sacrificed and dissected. Organs were imaged for bioluminescence ex vivo using an IVIS imaging system.

### Results

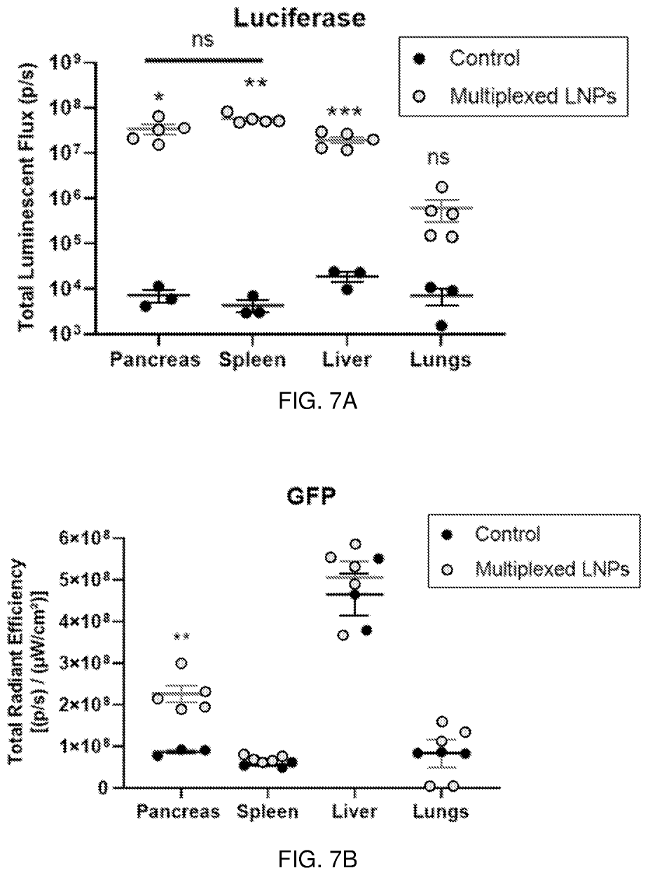

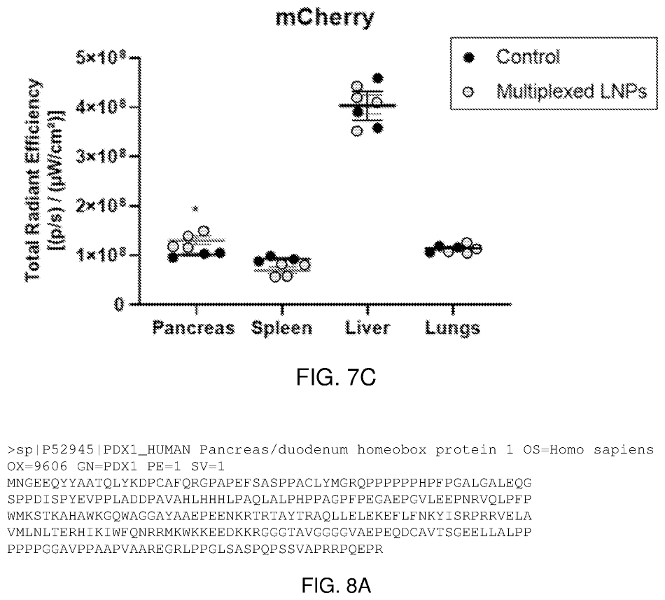

FIGS. 7A-7C show that mRNA lipid nanoparticles can simultaneously deliver three distinct mRNAs (encoding luciferase, GFP, and mCherry) to the pancreas. Although the lipid nanoparticles in this example were delivered intraperitoneally, effective delivery through other parenteral routes in equivalent doses, are contemplated, such as intradermal, intramuscular, subcutaneous, and intravenous routes, and oral routes.

### Example 5

### Materials and Methods

Lipid nanoparticle synthesis: The lipid nanoparticles were prepared as described in Example 2. The LNPs comprised 35% 306Oᵢ₁₀, 40% DOTAP, 22.5% cholesterol, and 2.5% C₁₄-PEG₂₀₀₀ PE and mRNA encoding luciferase.

In vivo experiments: The lipid nanoparticles were delivered to mice pre-treated with streptozotocin (Jackson Laboratory, Bar Harbor, ME) at a dosage of 0.5 mg mRNA/kg body weight by IP injection. Three hours later, mice were treated with D-luciferin for 15 min, then sacrificed and dissected. Organs were imaged for bioluminescence ex vivo using an IVIS imaging system. Data were analyzed using LivingImage software and plotted in GraphPad Prism.

### Results

As shown in FIG. 11, mRNA delivery to the pancreas was reduced when beta cells in the pancreas were depleted with streptozotocin (STZ).

### Example 6

### Materials and Methods

Lipid nanoparticle synthesis: The ionizable lipidoid 306Oᵢ₁₀ was combined with one of three helper lipids (DOTAP, 18:1 EPC, or DDAB), cholesterol, and C₁₄-PEG₂₀₀₀ PE in a 35:40:22.5:2.5 molar ratio in 100% ethanol. To formulate mRNA lipid nanoparticles, the lipid solution was combined 1:1 (vol/vol) with a solution of mRNA encoding firefly luciferase (10:1 lipidoid:mRNA w/w) suspended in 10 mM sodium citrate (pH 4) by vortexing. Lipid nanoparticles were diluted 1:1 (vol/vol) in PBS (pH 7.4) and further dialyzed against PBS for one hour.

In vivo experiments: The lipid nanoparticles were delivered to C57BL/6 mice (6-8 weeks, female) at a dosage of 0.5 mg mRNA/kg body weight by IP injection. Three hours later, mice were treated with D-luciferin for 15 min, then sacrificed and dissected. Organs were imaged for bioluminescence ex vivo using an IVIS imaging system. Data were analyzed using LivingImage software and plotted in GraphPad Prism.

### Results

The cationic helper lipids EPC and DDAB, like DOTAP as described in Example 2, were found to increase the fraction of the total signal that occurs in the pancreas (FIG. 12).

### Example 7

### Methods and Materials

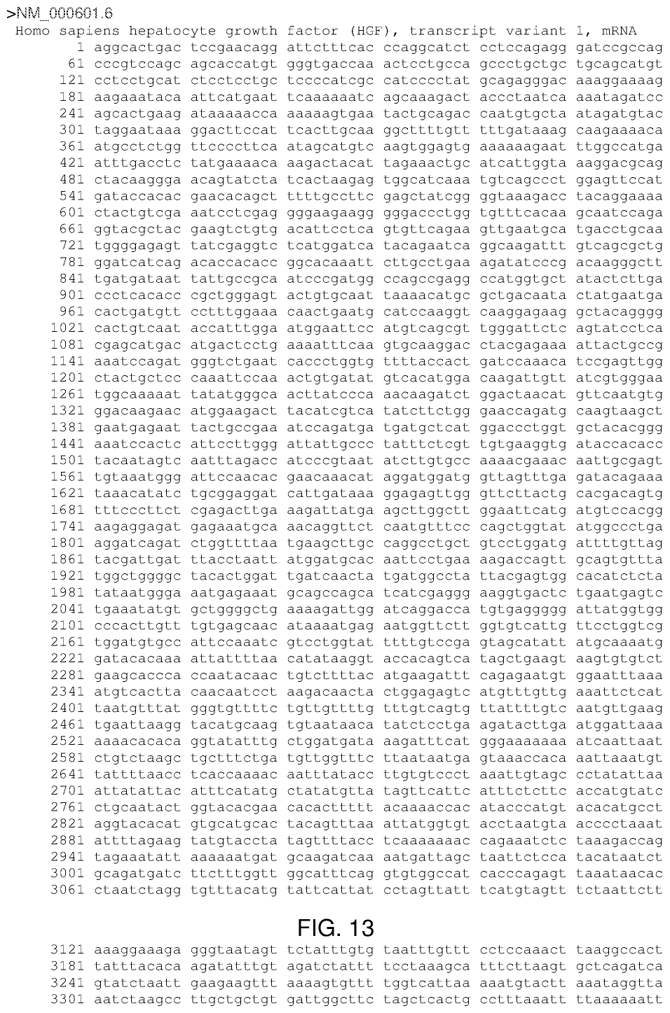

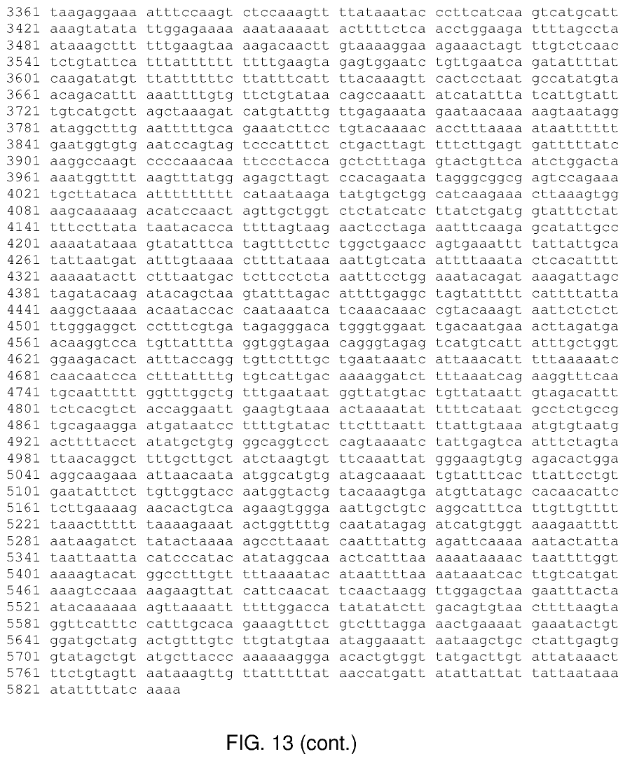

The delivery strategy described above in Examples 1-6 could be applied to a number of pancreatic diseases, including an application in type 1 diabetes. In this application, mRNAs encoding the transcription factors pancreatic and duodenal homeobox 1 (PDX1), MafA, and neurogenin-3 (Ngn3) could be delivered in combination to pancreatic acinar or alpha cells to induce conversion of the acinar or alpha cells to insulin-producing beta cells (Xiao, et al., “Endogenous Reprogramming of Alpha Cells into Beta Cells, Induced by Viral Gene Therapy, Reverses Autoimmune Diabetes,” Cell Stem Cell, 2018, 22:78-90; Cavelti-Weder, et al., “Reprogramming of Pancreatic Acinar Cells to Functional Beta Cells by In Vivo Transduction of a Polycistronic Construct Containing Pdx1, Ngn3, MafA in Mice.” Curr Protoc Stem Cell Biol. 2017, 40(1):4A-10). Additionally, mRNA encoding hepatocyte growth factor (HGF), as exemplified by HGF transcript variant 1 (e.g., FIG. 13, SEQ ID NO: 7), but including other functional variants, could be delivered to beta cells to increase their proliferation and resistance to diabetogenic stressors (see, e.g., Jimenez, et al., “In vivo genetic engineering of murine pancreatic beta cells mediated by single-stranded adeno-associated viral vectors of serotypes 6, 8, and 9.” Diabetologia. 2022, 54:1075-1086; Garcia-Ocana, et al., “Hepatocyte growth factor overexpression in the islet of transgenic mice increases beta cell proliferation, enhances islet mass, and induces milk hypoglycemia.” J Biological Chemistry. 1999, 275(2):1226-1232). HGF mRNA is commercially available, for example and without limitation, from phaRNA of Houston TX or Creative Biogene of Shirley, NY.

While the present invention is described with reference to several distinct embodiments, those skilled in the art may make modifications and alterations without departing from the scope and spirit. Accordingly, the above detailed description is intended to be illustrative rather than restrictive.

1. A method of delivering a therapeutic agent to a pancreas of a patient, comprising administering to a patient a composition comprising a lipid-containing particle, such as a lipid nanoparticle, comprising a therapeutic agent, the lipid-containing particle comprising:
a cationic helper lipid;
cholesterol or a derivative thereof;
a PEG-based compound; and
an ionizable lipidoid.

2. The method of claim 1, wherein the lipid particle comprises:
from 10 to 50 mole percent (mol %) of the cationic helper lipid;
from 10 to 46.5 mol % of the cholesterol or a derivative thereof;
from 1.25 to 2.5 mol % of the PEG-based compound; and
from 20 to 45 mol % of the ionizable lipidoid.

3. (canceled)

4. The method of claim 1, wherein the cationic helper lipid is one or more of 1,2-di-O-octadecenyl-3-trimethylammonium propane (DOTMA); a glycero-ethylphosphocholine (EPC) lipid, such as 12:0-sn-glycero-3-ethylphosphocholine (12:0 EPC), 14:0 EPC, 16:0 EPC, 18:0 EPC, 18:1 EPC, 16:0-18:1 EPC, and 14:1-EPC; dimethyldioctadecylammonium (DDAB); a trimethylammonium-propane (TAP) lipid, such as 14:0 TAP, 18:0 TAP, or 18:1 TAP (DOTAP); 3β-[N-(N′,N′-dimethylaminoethane)-carbamoyl]cholesterol hydrochloride (DC-chol); N⁴-cholesteryl-spermine (GL67); 1,2-dioleyloxy-3-dimethylaminopropane (DODMA); 1,2-dioleoyl-3-dimethylammonium-propane (DODAP); and N1-[2-((1S)-1-[(3-aminopropyl)amino]-4-[di(3-amino-propyl)amino] butylcarboxamido)ethyl]-3,4-di[oleyloxy]-benzamide (MVL5).

5. (canceled)

6. The method of claim 1, wherein the PEG-based compound is a PEGylated C₁₀-C₁₆ fatty acid-containing compound.

7. The method of claim 6, wherein the PEGylated fatty-containing compound is C₁₄-PEG₂₀₀₀ PE.

8. The method of claim 1, comprising cholesterol.

9. The method of claim 1, wherein the ionizable lipidoid is 306Oᵢ₁₀, 200Oᵢ₁₀, 514O_{6,10}.

10. The method of claim 1, wherein the ionizable lipidoid is 306Oᵢ₁₀.

11. The method of claim 1, wherein the therapeutic agent is anionic or polyanionic.

12. The method of claim 11, wherein the therapeutic agent is a nucleic acid.

13. The method of claim 12, wherein the nucleic acid is an RNA.

14. The method of claim 13, wherein the RNA is an RNA reagent chosen from an RNAi reagent, a dsRNA, an siRNA, an shRNA, a miRNA, an antisense RNA, an mRNA, a guide RNA (gRNA), a long non-coding RNAs (IncRNA), a base editing gRNA(beRNA), a prime editing gRNA (pegRNA), or a transfer RNA (tRNA).

15. (canceled)

16. The method of claim 1, wherein the lipid-containing particle comprises:
DOTAP as the cationic helper lipid;
cholesterol;
C₁₄-PEG₂₀₀₀ PE as the PEGylated fatty acid-containing compound; and
306Oᵢ₁₀ as the ionizable lipidoid.

17. The method of claim 1, wherein the lipid-containing particle comprises the ionizable lipidoid, DOTAP, cholesterol, and C₁₄-PEG₂₀₀₀ PE in an ionizable lipidoid:DOTAP:cholesterol:C₁₄-PEG₂₀₀₀ PE molar ratio of less than or equal to 20 to less than or equal to 55: less than or equal to 10 to less than or equal to 60: less than or equal to 10 to less than or equal to 50: less than or equal to 1 to less than or equal to 2.5.

18. The method of claim 17, wherein the lipid-containing particle comprises the ionizable lipidoid, DOTAP, cholesterol, and C₁₄-PEG₂₀₀₀ PE in an ionizable lipidoid: DOTAP:cholesterol:C₁₄-PEG₂₀₀₀ PE molar ratio of approximately 35:40:22.5:2.5.

19. The method of claim 1, wherein the lipid-containing particle is delivered to the patient parenterally or orally.

20. The method of claim 19, wherein the lipid-containing particle is delivered to the patient intravenously, intraperitoneally, intramuscularly, subcutaneously, or intradermally.

21. (canceled)

22. The method of claim 1, wherein the lipid-containing particle comprises:
EPC as the cationic helper lipid;
cholesterol;
C₁₄-PEG₂₀₀₀ PE as the PEGylated fatty acid-containing compound; and
306Oᵢ₁₀ as the ionizable lipidoid.

23. The method of claim 22, wherein the EPC is 18:1 EPC.

24. The method of claim 1, wherein the lipid-containing particle comprises:
DDAB as the cationic helper lipid;
cholesterol;
C₁₄-PEG₂₀₀₀ PE as the PEGylated fatty acid-containing compound; and
306Oᵢ₁₀ as the ionizable lipidoid.


SEQUENCE LISTING (7 sequences)

SEQ ID NO: 1 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 2 - DNA, 2603 bp, Homo sapiens
gagatcagtgcggagctgtcaaagcgagcaggggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgtttt

SEQ ID NO: 3 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 4 - DNA, 2669 bp, Homo sapiens
agccgtgggaggcggggccggccggcggcgcgggtggggcgcgggagcggtcccggagcagcccgaggcggcggccgcggggaggaggcggcgacgcgggcccggggtcgcccgagacacctggccagcggtgcccctagcgcgccgccccggagttgaccacgtgaaacttttccctgcgcccctcggcgccgccgccccgcgccggcgcccccccgcccccgccgggaccgccgcccgcggggagcaggggggggagaggcctgcagctccccccccactcccacgccgcccgtcggggcgcggccgggcgcgggccccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccatcaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggaccccgagcccgcgccgccgtcgccggggacaagttcgcgcaggcctctcggggcctcggctcggactccgcggtacaggacgtggacaccaggcccggcccggccgtgctggccccggtgccaagtctgcgggcgcggggctggaggccccttcgctcccggtccccgttcgcgcgcgtcggcccgggtcgccgtcctgaggttgagcggagaacggtgatttctaaggaaacttgagccaggtctaacttctttccaagcgtccgcttgtacatacgttgaacgtggttctccgttcccaccttcgccctgccagcctagagggaccgcgctgccgtcccttcccgggtggcccctgcctgcccccgccctccttcgttctcttctcagcctccctttccttgccttttttaacttcccctccccgttttaaaatcggtcttattttcgaagtatttataattattatgcttggtgattagaaaagaaaaccttggaggaagccccttctttccccagccggggtccgccctcagtcgcgagtcacagcatgagtcgctcgccaggaggggcccggcccctgcctgccccctccccgcttgcccccgaccctgctaccggcgttccttggaggtcgaagccagggacgtcacccgtgctgtgtccaggcctgctgtcctactatgctcaaccgggggtggggggaggggggtgagtcctgtgctcagtcgggtgggggctggcccggatcccgagctgctgtctctctatgcaccagaacatatctgtaactcctggggaaatacatcttgttttaaccttcaagagaagtgaaagaaaaaagtaatgcacagtatttctagcagaaaatttttttttttaagaggaggcttgggccagagccttctggcatggggcgggtggagaaagtgtttttattttaatttaaattgtgtttcgttttgtttgtggaatctttctttaatgcttcgtcgctctttggactagccgggagagagggcgaggaggcgggtgctccaggccctgtaggctgggccaggcgcctgggggatctgcccgttttcggaggccctcaggggccatcagtgggattccagccgctccacacccctcccctgagcactcggagtggaaggcgcgccgactcgttgaaagttttgttgtgtagttggttttcgttgagttcttttttcatttgctacgaaactgagaaaaagaaaaaaatacacaaaataaatctgttcagatccaa

SEQ ID NO: 5 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 6 - DNA, 1560 bp, Homo sapiens
gctcaggaaatccctgcggtctcaccgccgcgcctcgagagagagcgtgacagaggcctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgaaaggacctgtctgtcgctgggctgtgggtgctaagggtaagggagagggagggagccgggagccgtagagggtggccgacggcggcggccctcaaaagcacttgttccttctgcttctccctggctgacccctggccggcccaggcctccacgggggcggcaggctgggttcattccccggccctccgagccgcgccaacgcacgcaacccttgctgctgcccgcgcgaagtgggcattgcaaagtgcgctcattttaggcctcctctctgccaccaccccataatctcattcaaagaatactagaatggtagcactacccggccggagccgcccaccgtcttgggtcgccctaccctcactcaagtctgtctgcctctcagtctcttaccacccctcctccaatgtgattcaatccaatgtttggtctctcagcgcttactccccttgccttgctccaaagacgctgccgatctgctctactcccaatcaggtccgggatttcagggcgcctcactctgccttaaagccacgaaggcgaccctctgccttctcctcgtgcacttttcggagccattgccctcccggggcggaagaccaggctgtgaactgggaaagcgctagcccggccagggagcatctccccagcctccctgcgaactgcgcctgaaacgtgagctgcgctgcaggtgcctggagcac

SEQ ID NO: 7 - DNA, 5834 bp, Homo sapiens
aggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttcatatgctatatgttatagttcattcatttctcttcaccatgtatcctgcaatactggtacacgaacacactttttacaaaaccacatacccatgtacacatgcctaggtacacatgtgcatgcactacagtttaaattatggtgtacctaatgtaacccctaaatattttagaagtatgtacctatagttttacctcaaaaaaaccagaaatctctaaagaccagtagaaatattaaaaaatgatgcaagatcaaaatgattagctaattctccatacataatctgcagatgatcttctttggttggcatttcaggtgtggccatcacccagagttaaataacacctaatctaggtgtttacatgtattcattatcctagttatttcatgtagtttctaattcttaaaggaaagagggtaatagttctatttgtgtaatttgtttcctccaaacttaaggccacttatttacacaagatatttgtagatctattttcctaaagcatttcttaagtgctcagatcagtatctaattgaagaagtttaaaagtgttttggtcattaaaaatgtacttaaataggttaaatctaagccttgctgctgtgattggcttctagctcactgcctttaaattttaaaaaatttaagaggaaaatttccaagtctccaaagttttataaatacccttcatcaagtcatgcattaaagtatatattggagaaaaaaataaaaatacttttctcaacctggaagattttagcctaataaagcttttttgaagtaaaagacaacttgtaaaaggaaagaaactagtttgtctcaactctgtattcatttattttttttttgaagtagagtggaatctgttgaatcagatattttatcaagatatgtttattttttcttatttcattttacaaagttcactcctaatgccatatgtaacagacatttaaattttgtgttctgtataacagccaaattatcatatttatcattgtatttgtcatgcttagctaaagatcatgtatttgttgagaaatagaataacaaaaagtaataggataggctttgaatttttgcagaaatcttcctgtacaaaacacctttaaaaataattttttgaatggtgtgaatccagtagtcccatttctctgacttagttttcttgagtgatttttatcaaggccaagtccccaaacaattccctaccagctctttagagtactgttcaatctggactaaaatggttttaagtttatggagagcttagtccacagaatatagggcggcgagtccagaaatgcttatacaatttttttttcataataagatatgtgctggcatcaagaaacttaaagtggaagcaaaaagacatccaactagttgctggtctctatcatcttatctgatggtatttctattttccttatataatacaccattttagtaagaactcctagaaatttcaagagcatattgccaaaatataaagtatatttcatagtttcttctggctgaaccagtgaaattttattattgcatattaatgatatttgtaaaacttttataaaaattgtcataattttaaatactcacattttaaaaatacttctttaatgactcttcctctaaatttcctggaaatacagataaagattagctagatacaagatacagctaagtatttagacattttgaggctagtatttttcattttattaaaggctaaaaacaataccaccaataaatcatcaaacaaaccgtacaaagtaattctctctttgggaggctcctttcgtgatagagggacatgggtggaattgacaatgaaacttagatgaacaaggtccatgttattttaggtggtagaacagggtagagtcatgtcattatttgctggtggaagacactatttaccaggtgttctttgctgaataaatcattaaacatttttaaaaatccaacaatccactttattttgtgtcattgacaaaaggatcttttaaatcagaaggtttcaatgcaatttttggtttggctgtttgaataatggttatgtactgttataattgtagacattttctcacgtctaccaggaattgaagtgtaaaactaaaatatttttcataatgcctctgccgtgcagaaggaatgataatccttttgtatacttctttaattttattgtaaaatgtgtaatgacttttacctatatgctgtgggcaggtcctcagtaaaatctattgagtcaatttctagtattaacaggcttttgcttgctatctaagtgtttcaaattatgggaagtgtgagacactggaaggcaagaaaattaacaataatggcatgtgatagcaaaattgtatttcacttattcctgtgaatatttcttgttggtaccaatggtactgtacaaagtgaatgttatagccacaacattctcttgaaaagaacactgtcaagaagtgggaaattgctgtcaggcatttcattgttgtttttaaacttttttaaaagaaatactggttttgcaatatagagatcatgtggtaaagaattttaataagatcttatactaaaaagccttaaatcaatttattgagattcaaaaaatactattataattaattacatcccatacatataggcaaactcatttaaaaaataaaactaattttggtaaaagtacatggcctttgtttttaaaatacataattttaaaataaatcacttgtcatgataaagtccaaaaagaagttatcattcaacattcaactaaggttggagctaagaatttactaatacaaaaaaagttaaaattttttggaccatatatatcttgacagtgtaacttttaagtaggttcatttccatttgcacagaaagtttctgtctttaggaaactgaaaatgaaatactgtggatgctatgactgtttgtcttgtatgtaaataggaaattaataagctgcctattgagtggtatagctgtatgcttacccaaaaaagggaacactgtggttatgacttgtattataaactttctgtagttaataaagttgttatttttataaccatgattatattattattattaataaaatattttatcaaaa